(19) United States
(12) Patent Application Publication
(10) Pub. No.: US 2015/0088151 A1
(43) Pub. Date: Mar. 26, 2015

(54) ELONGATED MEMBER FOR MEDICAL USE AND CONNECTING MEMBER

(71) Applicant: TERUMO KABUSHIKI KAISHA, TOKYO (JP)

(72) Inventor: TOMONORI HATTA, KANAGAWA (JP)

(21) Appl. No.: 14/495,333

(22) Filed: Sep. 24, 2014

(30) Foreign Application Priority Data
Sep. 25, 2013 (JP) 2013197932

(51) Int. Cl. A61B 17/00

(52) U.S. Cl. CPC A61B 17/00234
USPC 606/108

(57) ABSTRACT

An elongated member for medical use that is used to perform a procedure in a pulmonary airway. The elongated member demonstrates enhanced reachability within the pulmonary airway and is fabricated by connecting plural hollow members different from each other in lumen diameter, outer diameter and physical properties. This configuration advantageously eliminates the formation of a step or ridge that would impede the smooth movement or manipulation of a procedure instrument that is advanced or retreated within the lumen of the elongated member.

### CROSS REFERENCE TO RELATED APPLICATIONS

This application claims priority to Japanese National Application No. 2013-197932, filed on Sep. 25, 2013. The entire contents of which is incorporated herein by reference.

### BACKGROUND

The present disclosure relates to an elongated member for medical use particularly for performing a procedure in a pulmonary airway, and also relates to a connecting member used with elongated member.

An elongated member for medical use is known for use in performing various kinds of procedures in a pulmonary airway of a living body, such as diagnosis of the disease state of a patient, treatment for a lesion, and delivery of various kinds of media into a biological organ.

For example, an elongated member is used to assist the movement of a medical device for achieving a diagnostic image such as an endoscopic image when the medical device is moved to a lesion in some cases. Furthermore, it is used to guide a medical instrument such as a biopsy instrument to a lesion in a manipulation of, for example, cutting a biological tissue. The elongated member is often formed integrally with a catheter device and may be used for delivery of various kinds of medications into a living body, for suction of a body fluid or other fluids and/or foreign substances existing in a living body.

To enhance the reachability of the elongated member to the peripheral side of bronchi in performing various kinds of procedures in a pulmonary airway, it is preferable to form the distal part of the elongated member with a smaller diameter. With regard to the delivery of various kinds of procedure instruments, medications, and other substances toward the peripheral side of bronchi via the inner cavity (lumen) of the elongated member from the proximal side to the distal side, it is preferable that the proximal side of the elongated member has a larger lumen diameter for ease of the delivery. Furthermore, in view of the operability of the elongated member in the pulmonary airway, it is advantageous for the distal side to be endowed with flexibility and the proximal side to be endowed with pushability (i.e., slightly more rigid/inflexible in order to effect transmissibility of a pushing force).

Japanese Patent Laid-open No. 2012-196389 discloses an elongated member for medical use that is given a tapered shape in which the inner diameter and outer diameter become smaller toward the distal side and is so formed such that the physical properties (hardness and rigidity) of the respective parts are different in the axial line direction.

With regard to a method for manufacturing the elongated member, a method has been employed in which plural outer layer members having different physical properties are deposited as covering members at different positions, in the axial line direction, on a hollow base member composed of a single material. By this method, the elongated member is given such a physical property that the hardness becomes lower toward the distal side.

### SUMMARY OF THE DISCLOSURE

The related art described in Japanese Patent Laid Open No. 2012-196389 can provide an elongated member that has desired inner diameter and outer diameter and in which flexibility is ensured on the distal side. However, because the elongated member described in Japanese Patent Laid Open No. 2012-196389 is formed by using the base member composed of a single material, the elongated member has physical properties according to the material of this base member. Therefore, in the case of forming an elongated member in such a manner that the physical properties of the distal side of the elongated member are greatly different from those of the proximal side, specifically in the case of attempting to facilitate more flexible deformation of the distal side and endow the proximal side with higher pushability, it is difficult to endow the elongated member with desired physical properties by merely depositing the outer layer members as covering members because the physical properties of the elongated member depend on the physical properties of the base member.

As a countermeasure against the above-described problem, provision of an elongated member suitable for a procedure in a pulmonary airway would be enabled by manufacturing the elongated member by connecting plural hollow members different from each other in lumen diameter, outer diameter, and physical properties. However, if the configuration in which hollow members of different connected lumen diameters is employed, a step or “ridge” is formed at the joint part across which the hollow members are connected to each other. If such a step is formed, when a predetermined medical device, procedure instrument, or the like is made to advance or retreat in the lumen of the elongated member, it gets caught or impeded on the step and the smooth movement thereof is hindered. Moreover, a break in the medical device, procedure instrument, or the like can occur. Also in the case of performing suction of a body fluid or the like, or the discharge of a medication or the like by use of the elongated member, the smooth flow of various kinds of fluid is hindered. As a result work efficiency is lowered and there is a delay in the performance of operation.

Accordingly, an intention of the present disclosure is to provide an elongated member for medical use that has excellent operability in a pulmonary airway and prevents a step from being formed at a connection part across which plural members having different lumen diameters and the outer diameter are connected so as to thereby enable the smooth movement of various kinds of members, fluids, and the like in the lumen. This disclosure further describes a connecting member used for the elongated member for medical use.

In one embodiment an elongated member for medical use is contemplated that is used to perform a procedure in a pulmonary airway. The elongated member includes the following elements: a first member disposed on a distal side, a first lumen extending along an axial direction formed in the first member; a second member in which a second lumen is formed, the second lumen extends along the axial direction and has a larger inner diameter than the first lumen is formed, the second member having a larger outer diameter than the first member and being disposed on a proximal side of the first member; and a connecting member having a distal insertion hole into which a proximal part of the first member is insertable, a proximal insertion hole into which a distal part of the second member is insertable, and a third lumen that communicates with the distal insertion hole and the proximal insertion hole, a diameter of the third lumen gradually decreasing from the proximal side toward the distal side from the diameter of the second lumen to the diameter of the first lumen, the connecting member connecting the first member and the second member.

In a second embodiment, the elongated member for medical use is provided wherein the proximal part of the first member is so formed as to be capable of being connected to the distal insertion hole of the connecting member through fitting, and the distal part of the second member is so formed as to be capable of being connected to the proximal insertion hole of the connecting member through fitting.

In another embodiment, the elongated member for medical use is provided wherein a distal tapered part whose outer diameter gradually decreases toward the distal side is formed at a distal part of the connecting member, and a proximal tapered part whose outer diameter gradually decreases toward the proximal side is formed at a proximal part of the connecting member.

In an additional embodiment, there is provided the elongated member for medical use, further including a covering member that is so disposed as to cover an outer surface of the first member, an outer surface of the connecting member, and an outer surface of the second member.

The instant disclosure also provides a connecting member to assemble an elongated member for medical use that is used to perform a procedure in a pulmonary airway by connecting the proximal side of a first member in which a first lumen extending along the axial direction is formed to a second member in which a second lumen that extends along the axial direction and has a larger inner diameter than the first lumen is formed, the second member having a larger outer diameter than the first member. The connecting member includes the following elements: a distal insertion hole into which a proximal part of the first member is insertable; a proximal insertion hole into which a distal part of the second member is insertable; and a third lumen that communicates with the distal insertion hole and the proximal insertion hole, the diameter of the third lumen gradually decreasing from the proximal side toward the distal side from the diameter of the second lumen to the diameter of the first lumen.

According to the instant disclosure, the elongated member in one embodiment can be formed by connecting the first member and the second member that are different from each other in the lumen diameter, outer diameter, and physical properties. Therefore, by arbitrarily selecting members suitable for the dimensions and physical properties of the respective parts of the elongated member, the following elongated member for medical use suitable to be used in the pulmonary airway can be provided. Specifically, the distal side is given a configuration that has a small diameter and is excellent in flexibility and the proximal side is endowed with pushability. Moreover, the lumen diameter on the proximal side is set large. In the connecting member used to connect the first member and the second member, the lumen is formed whose diameter continuously changes from the proximal side toward the distal side according to the difference in the lumen diameter between the respective members. Therefore, the respective members can be connected to each other without forming a step at the connection part as the joint of the respective members. This can provide the elongated member for medical use with which the smooth movement of various kinds of members, fluids, and so forth in the lumen is realized.

The instant disclosure further provides an elongated member wherein the first member and the second member can be connected by inserting the proximal part of the first member into the distal insertion hole of the connecting member and inserting the distal part of the second member into the proximal insertion hole of the connecting member. This facilitates manufacturing and reduces manufacturing cost.

In accordance with the instant disclosure, when operation of moving the elongated member in a pulmonary airway is carried out, friction between the connecting member and the inner wall surface of the pulmonary airway can be reduced by the respective tapered parts each formed at a respective one of the distal part and the proximal part of the connecting member and the pushability of the elongated member can be enhanced. Furthermore, it is possible to prevent the occurrence of damage on the inner wall surface of the pulmonary airway when pushing operation is carried out.

The respective parts of the elongated member can, for example, be protected by the covering member. This can favorably prevent causing of a break in the elongated member when it is used. Furthermore, when various kinds of fluids are made to flow by using the elongated member, the occurrence of the leakage of the fluid from the elongated member can be favorably prevented.

According to the instant disclosure in accordance with the above-described embodiments, it is possible to provide the connecting member that is used to assemble the elongated member for medical use including the first member and the second member as its constituent elements and allows the respective members to be connected without forming a step at the connection part across which the respective members are connected.

### BRIEF DESCRIPTION OF THE DRAWINGS

FIG. 1 is an outline perspective view of a medical instrument according to an embodiment of the present disclosure.

FIGS. 2A and 2B diagrammatically illustrate the medical instrument and a connecting member according to one embodiment; FIG. 2A being a partial sectional view showing the overall configuration of the medical instrument in a simplified manner, and FIG. 2B being an enlarged sectional view showing the connecting member.

FIG. 3 is an enlarged sectional view of the distal part of the medical instrument according to the embodiment.

FIGS. 4A and 4B are enlarged views showing sections of the respective parts of the medical instrument according to one embodiment; FIG. 4A being a sectional view along line 4A-4A in FIG. 3, and FIG. 4B being a sectional view along line 4B-4B in FIG. 3.

FIGS. 5A and 5B are enlarged views showing sections of the respective parts of the medical instrument according to the embodiment; FIG. 5A being a sectional view along line 5A-5A in FIG. 3, and FIG. 5B being a sectional view along line 5B-5B in FIG. 3.

FIGS. 6A and 6B diagrammatically illustrate a first member included in an elongated member according to one embodiment; FIG. 6A being a partial enlarged view showing part of the first member, FIG. 6B being an enlarged diagram showing a curving mechanism formed in the first member

FIG. 7 is a diagrammatic example of use of the elongated member for medical use according to one embodiment and schematically illustrates a state in which the elongated member for medical use is introduced into a pulmonary airway.

FIG. 8 is a diagrammatic example of use of the elongated member for medical use according to one embodiment and schematically illustrates state in which the distal end of the elongated member for medical use is moved toward the peripheral side of the pulmonary airway from the state shown in FIG. 7.

FIG. 9 is a diagrammatic example of use of the elongated member for medical use according to one embodiment and schematically illustrates a state in which the distal part of the elongated member for medical use is made to reach a respiratory bronchiole.

FIGS. 10A and 10B illustrate a modification example of the elongated member for medical use according to one embodiment; FIG. 10A being a diagram schematically showing an example of use of a medical device according to a comparative example, and FIG. 10B being a diagram schematically showing an example of use of an elongated member according to the modification example.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### DETAILED DESCRIPTION OF THE PREFERRED EMBODIMENTS

An embodiment of the present disclosure will be described below with reference to the drawings. The dimensional ratio of the drawings is exaggerated for convenience of description and is different from the actual ratio in some cases.

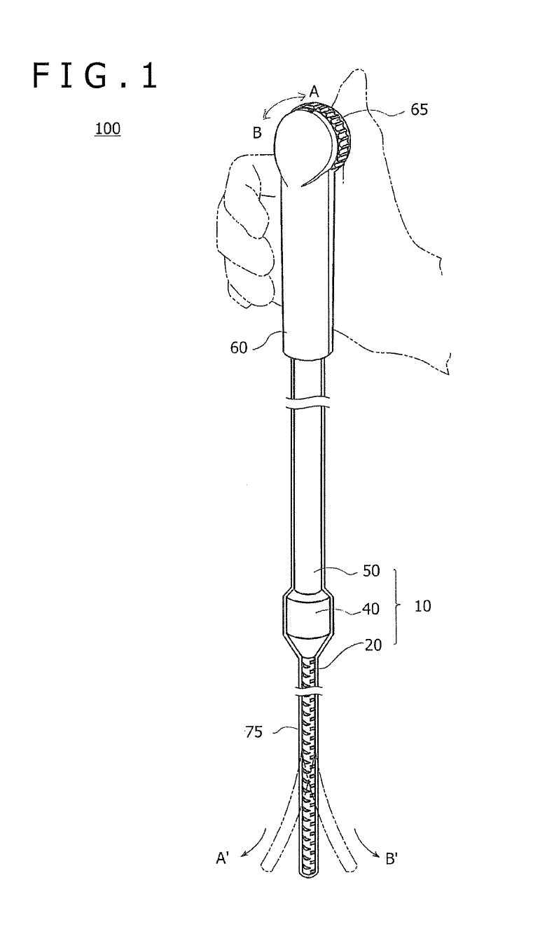

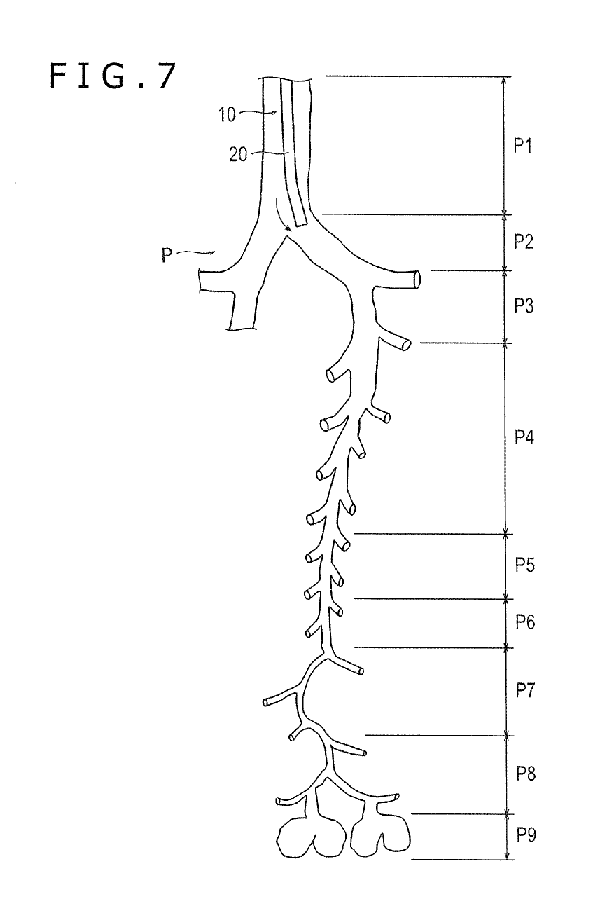

FIGS. 1 to 6B are illustrated the configuration of the respective parts of an elongated member for medical use according to one embodiment. FIGS. 7 to 9 explain an example of use of the elongated member for medical use according to one embodiment.

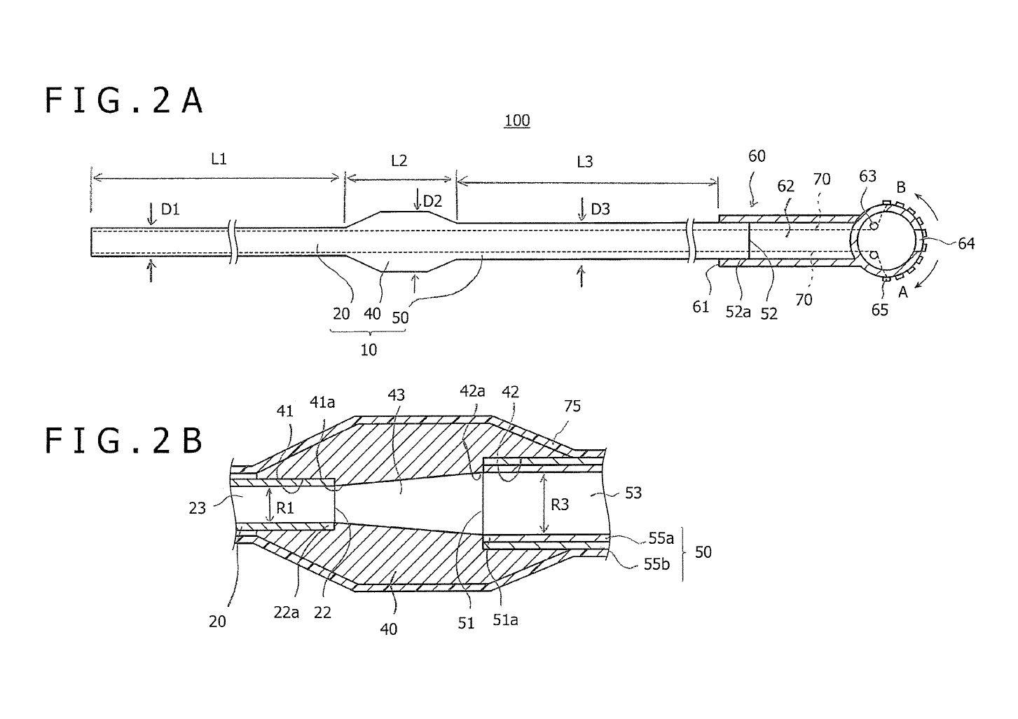

In the following description, the lower side in FIG. 1 and the left side in FIG. 2A will be referred to as the “distal side of the elongated member.” The upper side in FIG. 1 and the right side in FIG. 2A will be referred to as the “proximal side of the elongated member.” The vertical direction in FIG. 1 and the horizontal direction in FIG. 2A will be referred to as the “axial direction of the elongated member.”

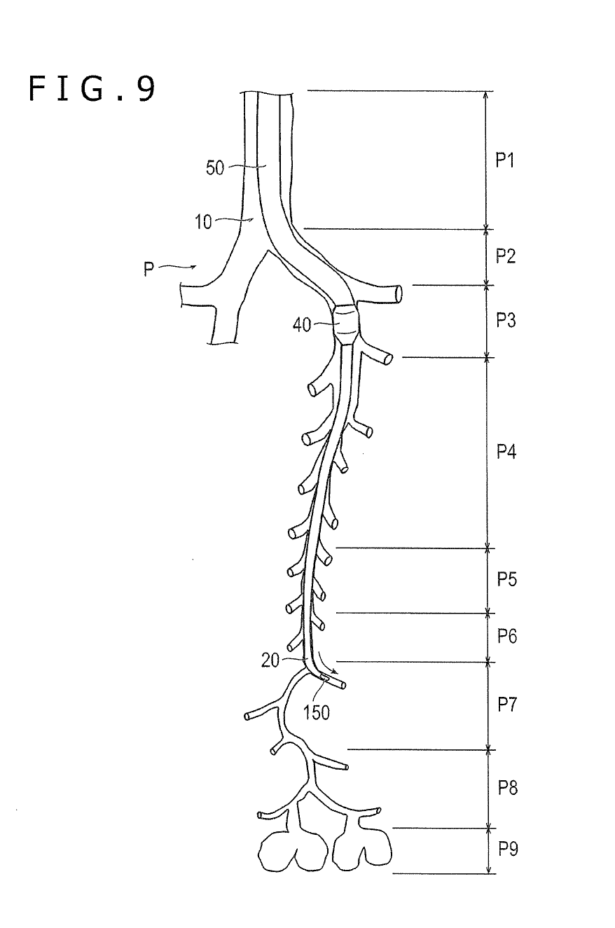

Referring to FIG. 1, an elongated member 10 for medical use according to the present embodiment (hereinafter, referred to simply as the “elongated member”) has an outer shape extended along the axial direction and its distal side is formed as a member having the ability to curve. The elongated member 10 can be used to perform various procedures in a pulmonary airway P (see FIG. 7). For example, elongated member 10 can be used for delivery of an imaging device such as an endoscope, delivery of a biopsy instrument for obtaining a biological tissue, and delivery of a medication and suction of a body fluid, such as but not limited to, mucus. Furthermore, it is also possible for the elongated member 10 to be used as a constituent member or the like that forms various kinds of access devices, the insertion part of a flexible endoscope, or the shaft part of a balloon catheter or the like. In the description of the present embodiment, the elongated member 10 will be explained through an example in which it is applied to a medical instrument 100 (access device) used to guide a predetermined procedure instrument 150 for medical use to a desired position in the pulmonary airway P as shown in FIG. 9.

As used herein throughout, pulmonary airway is generally considered to include the branched bronchi that extend from the lower trachea into the individual lungs, as well as other airways that are a part of the respiratory and pulmonary system.

The overall configuration of the elongated member according to the present embodiment will now be described.

As shown in FIGS. 1, 2A, and 2B, the elongated member 10 has a first member 20 disposed on the distal side, a second member 50 disposed on the proximal side of the first member 20, and a connecting member 40 that connects the first member 20 and the second member 50.

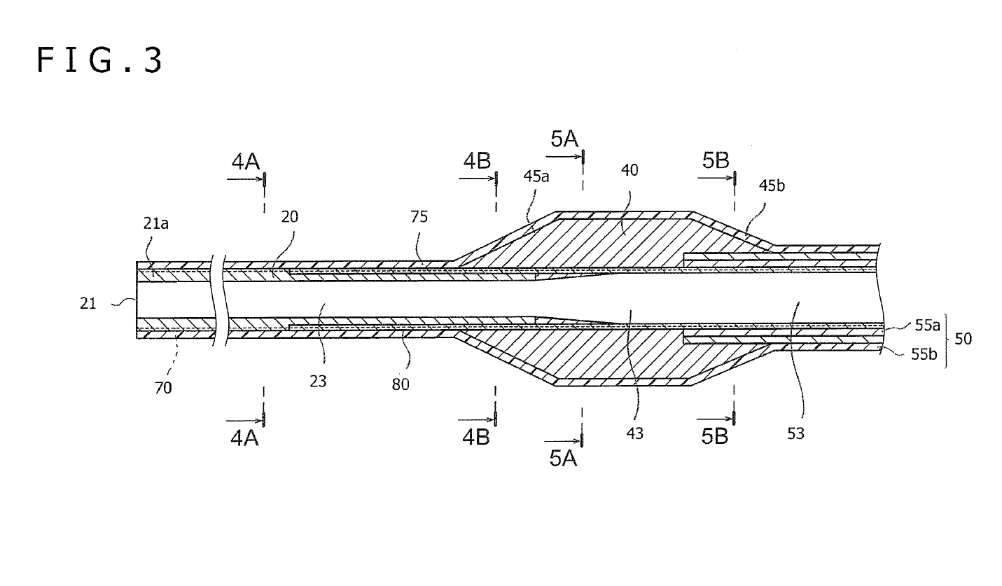

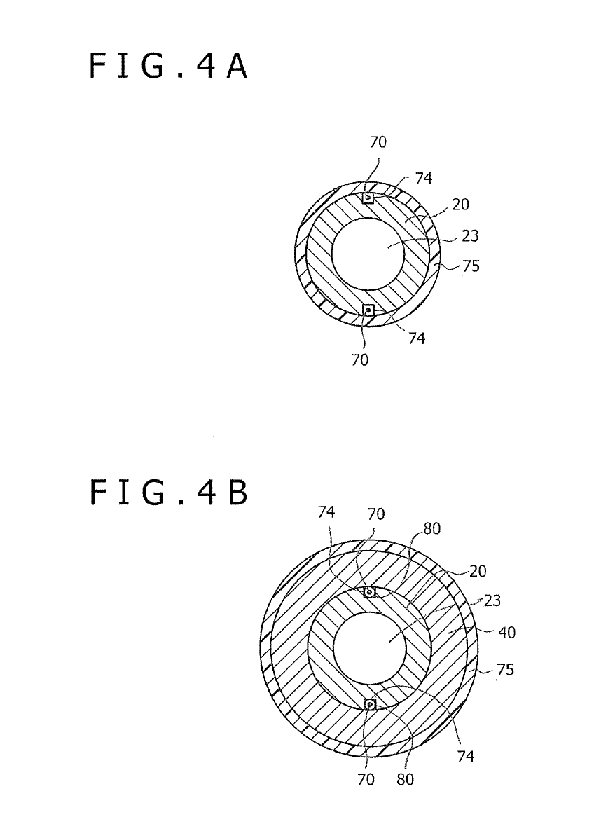

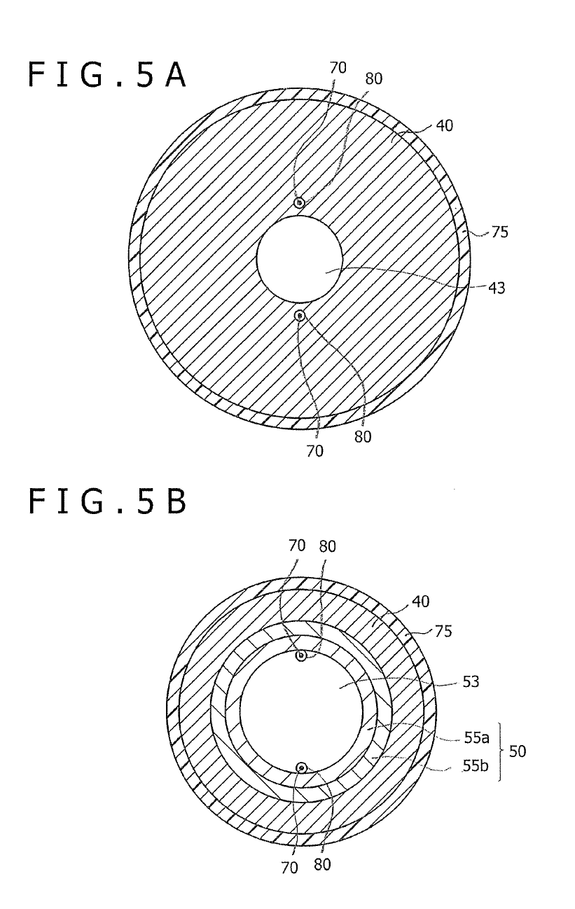

As shown in FIGS. 3, 4A, and 4B, in the first member 20, a lumen (equivalent to the first lumen) 23 extending along the axial direction is formed. Furthermore, as shown in FIGS. 3 and 5B, in the second member 50, a lumen (equivalent to the second lumen) 53 that extends along the axial direction and has a larger diameter than the lumen 23 of the first member 20 is formed. The second member 50 is so formed as to have a larger outer diameter than the first member 20. As above, the first member 20 and the second member 50 are formed of, for example, hollow members in which the lumens 23 and 53 extend along the axial direction.

As shown in FIG. 2B, the connecting member 40 has a distal insertion hole 41 into which a proximal part 22a of the first member 20 is insertable, a proximal insertion hole 42 into which a distal part 51a of the second member 50 is insertable, and a lumen (equivalent to the third lumen) 43 that communicates with the distal insertion hole 41 and the proximal insertion hole 42. The diameter of the lumen 43 gradually decreases from the proximal side toward the distal side from the diameter of the lumen 53 of the second member 50 to the diameter of the lumen 23 of the first member 20.

As shown in FIGS. 1, 2A and 2B, a hand operating part 60 for operating the curving action of the first member 20 is provided on the proximal side of the second member 50 included in the elongated member 10. In the present embodiment, the first member 20 is so formed as to curve into directions intersecting the axial direction through predetermined operation at the hand operating part 60 (A′ and B′ in FIG. 1 indicate the curving action). This hand operating part 60 and the elongated member 10 form the medical instrument 100.

Referring to FIGS. 2A and 2B, the first member 20 included in the elongated member 10 is provided in order to enable settlement of the traveling direction of the elongated member 10 at each bifurcation of the pulmonary airway P. As shown in FIG. 7, in the pulmonary airway P of a living body, plural bifurcations exist from the central side (upper side in the diagram) to the peripheral side (lower side in the diagram). When performing various kinds of procedures by using the elongated member 10, movement of the distal end of the elongated member 10 toward a desired position in the pulmonary airway P is carried out. At this time, selecting an arbitrary course at each bifurcation is enabled by appropriately curving the first member 20. In the following description, the first member 20 will be referred to as the “curving portion 20” for convenience.

Referring to FIGS. 2A and 2B, the connecting member 40 included in the elongated member 10 connects the curving portion 20 and the second member 50 in the axial direction. Using this connecting member 40 allows the elongated member 10 to be formed by connecting plural members different in outer diameter, lumen diameter, physical properties, and so forth.

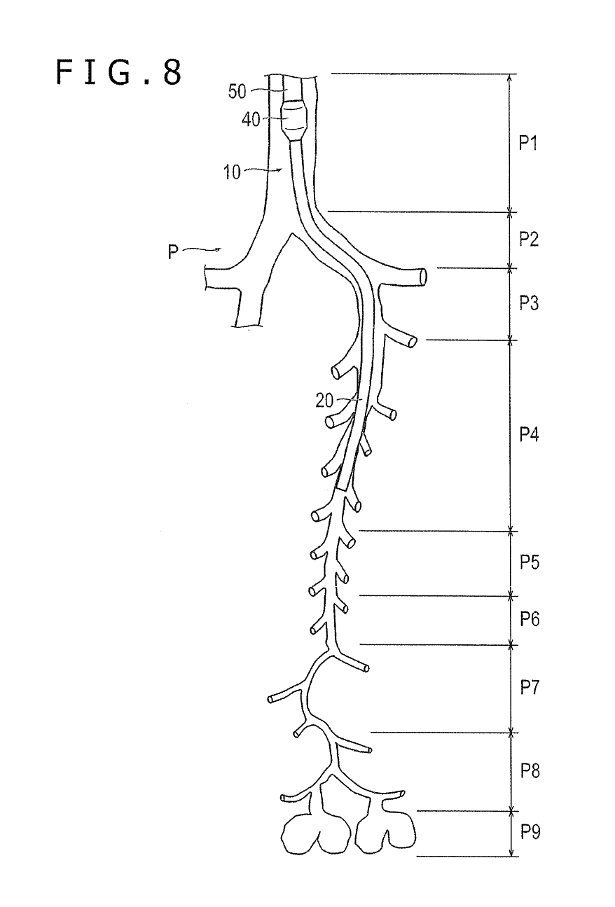

The connecting member 40 functions to assist the movement of the curving portion 20 in the pulmonary airway P. The curving portion 20 is so formed as to have such flexibility so as to curve in association with predetermined operation with the hand operating part 60 when the operation is carried out and form a substantially straight line shape when operation is not being carried out, i.e. when a load is not being applied thereto. Therefore, for example, as shown in FIGS. 8 and 9, when pushing the distal end of the elongated member 10 (distal end of the curving portion 20) into the pulmonary airway P is carried out, bending is caused in the curving portion 20 and the smooth movement of the elongated member 10 is hindered in some cases. By using the connecting member 40, which is so formed as to be different from the curving portion 20 in physical properties, outer shape, and so forth, as a constituent member of the elongated member 10, the smooth movement of the curving portion 20 in the pulmonary airway P is permitted. Additional details will be described herein below. In the following description, the connecting member 40 will be referred to as the “entry assisting portion 40” for convenience.

Referring now to FIGS. 2A and 2B, the second member 50 included in the elongated member 10 is extended with a predetermined length along the axial direction. The second member 50 is formed with a length that allows each of the curving portion 20 and the entry assisting portion 40 disposed on its distal side to reach desired positions in the pulmonary airway P (see FIG. 9). In the following description, the second member 50 will be referred to as the “main body portion 50” for convenience.

The structure of the pulmonary airway of a living body will be described in greater detail herein below.

With reference to FIG. 7, in the pulmonary airway P, the respective areas on the central side and the peripheral side have names different from each other. The names of the respective areas are as follows from the central side: trachea P1, main bronchus P2, lobar bronchus P3, bronchus P4, bronchiole P5, terminal bronchiole P6, respiratory bronchiole P7, alveolar duct P8, and alveolar sac P9. The diameters (sectional areas) of the respective areas P1 to P9 are different as shown in the diagram and the diameter becomes smaller toward the peripheral side.

When performing various procedures in the pulmonary airway P by using the elongated member 10, it is preferable that the curving portion 20 forming the distal part of the elongated member 10 is endowed with flexibility in order to allow the distal end of the elongated member 10 to easily reach a procedure-target site. Furthermore, as the curving portion 20, a component having an outer diameter smaller than the diameter of a target area in which the procedure-target site exists is selected. Meanwhile, it is preferable that the main body portion 50 disposed on the proximal side of the elongated member 10 is endowed with pushability to transmit a force that pushes the elongated member 10 into the pulmonary airway P to the distal side.

For example, as shown in FIGS. 8 and 9, when moving the elongated member 10 in an area on the peripheral side of the pulmonary airway P, the distal side of the elongated member 10 can be moved toward the peripheral side in such a manner as to follow the pulmonary airway P by carrying out operation in a state in which the outer surfaces of the respective parts of the elongated member 10 abut against the inner wall of the pulmonary airway P. If the proximal side is endowed with sufficient pushability, the pushing force can be sufficiently transmitted from the proximal side to the distal side when the above-described operation is carried out. This allows the distal part of the elongated member 10 to easily reach the target area located on the peripheral side of the pulmonary airway P. In the present embodiment, the periphery of the bronchiole P5 (peripheral airway), which is considered to be deeply tied to the occurrence of a lesion such as a COPD, and an area on the peripheral side, such as the respiratory bronchiole P7 close to the alveolar ducts P8 and the alveolar sacs P9, are deemed as the target area, and the dimensions and physical properties of the respective parts of the elongated member 10 are so set that various types of procedures can be efficiently performed in this target area.

The configurations of the respective parts of the elongated member will now be described.

As shown in FIGS. 2A, 2B, and 3, the curving portion 20 of the elongated member 10 is formed of a member in which a distal part 21a at which a distal opening 21 is formed, the proximal part 22a at which a proximal opening 22 is formed, and the lumen 23 that communicates with the distal opening 21 and the proximal opening 22 are formed.

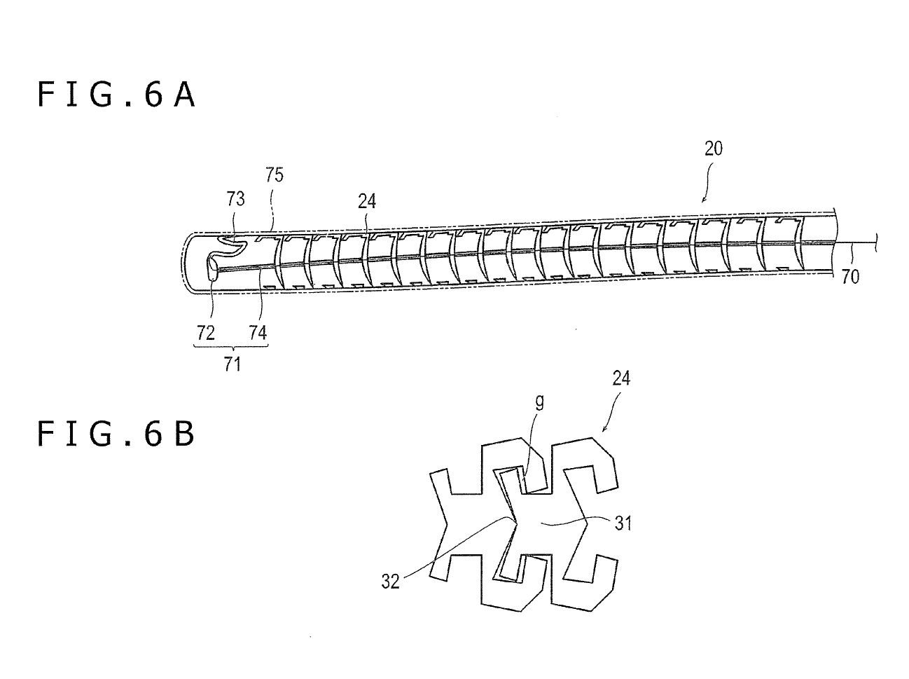

The curving portion 20 can be formed of, for example but not limited to, a hollow member that has flexibility and is so formed as to make a curving action in accordance with pushing operation of a predetermined operating member 70. In the present embodiment, the curving portion 20 is formed of a tubular member given flexibility through forming of grooves 24 (see FIGS. 6A and 6B).

The tubular member forming the curving portion 20 may be fabricated from, for example but not limited to, a metal material or a hard resin material. The metal material may be, for example, stainless steel or a nickel titanium alloy. For the resin material, the following materials can be used: PP (polypropylene), HDPE (high density polyethylene), rigid polyester such as PET (polyethylene terephthalate) and PBT (polybutylene terephthalate), rigid urethane, PI (polyimide), polystyrene, PEEK (polyether ether ketone), polyamide, polyetherimide, polyamide imide, modified polyphenylene ether, and polycarbonate.

As shown in FIG. 6A, the grooves 24 formed in the curving portion 20 are formed by making slits (cuts) having a predetermined shape in the outer surface (or inner surface) of the tubular member. The processing method for making the slits can be selected according to the characteristics of the material used as the curving portion 20 and is not particularly limited. For example, the slits can be formed by a known methods such as laser processing or etching processing.

Manipulation of the curving portion 20 can be carried out by operating the operating member 70 connected to the curving portion 20. As shown in FIG. 6A, the operating member 70 is attached to an attachment part 71 formed on the outer surface of the distal side of the curving portion 20. The attachment part 71 can be formed of, for example, a groove-shaped fixing part 72 disposed on the distal side of the curving portion 20 and an insertion groove 74 extended from the distal side of the curving portion 20 to the proximal side.

The operating member 70 can be formed of a push/pull member that is push/pull-operated along the longitudinal direction of the elongated member 10 to thereby deform the curving portion 20 into a curved or straight line shape. In the present embodiment, the push/pull member is formed of a wire having flexibility. However, it is also possible that the push/pull member can be formed of, for example, a string-shaped member or a plate material having flexibility.

The operating member 70 can be attached to the curving portion 20 by fixing the operating member 70 in the fixing part 72 and the fixing part of the operating member 70 in the insertion groove 74. By fixing the operating member 70 in this manner, the curving action of the curving portion 20 can be commenced by pulling the operating member 70. Furthermore, by loosening the pulling force, the curving action can be released and the curving portion 20 can be so deformed as to form a straight line shape.

As shown in FIG. 3, in the present embodiment, guide members 80 extended to range in the curving portion 20, the entry assisting portion 40, and the main body portion 50 are provided in order to allow the smooth push/pull operation of the operating member 70 and to prevent a break in the operating member 70 due to repeated push/pull operation. The guide member 80 is formed of a thin member into which the operating member 70 is insertable. The material used to form this guide member 80, may be the same material as that used to form the curving portion 20. Furthermore, the guide member 80 can be formed of, for example, a hollow member in which a lumen extends along the axial direction.

As shown in FIGS. 3, 4A, and 4B, the distal side of the guide members 80 can be disposed in the insertion grooves 74 that are formed in the curving portion 20. Furthermore, as shown in FIGS. 3 and 5B, the proximal side of the guide members 80 can be disposed in contact with the inner surface of the lumen 53 of the main body portion 50 for example. In addition, as shown in FIGS. 3 and 5A, the parts of the guide members 80 located between the proximal part and the distal part can be disposed so as to penetrate the entry assisting portion 40. As shown in FIG. 3, part of the operating member 70 on the distal side extends from the guide member 80 and this extended portion is fixed to the fixing part 72 formed in the curving portion 20 (see FIG. 6A).

In FIG. 6A, the insertion grooves 74 formed in the curving portion 20 can be formed at positions different from the positions at which the grooves 24 are formed in the elongated member 10 for example. In FIG. 6A, the insertion grooves 74 are disposed at positions offset by 90° in the circumferential direction from the positions at which the grooves 24 are formed (insertion grooves 74 are disposed at positions offset from each other by 180° in the circumferential direction). Disposing them in this manner can prevent the mutual interference of the respective grooves 24 and the operating member 70.

As shown in FIG. 6A, in the curving portion 20, a fixation assisting part 73 for fixing the operating member 70 to this curving portion 20 more strongly can be provided. The fixation assisting part 73 can be formed of a groove that is so formed as to extend with a predetermined length from the fixing part 72 along the circumferential direction of the curving portion 20. By carrying out the fixing in a state in which the operating member 70 is so positioned as to be locked to the fixation assisting part 73, the fixing strength can be enhanced.

With regard to the method for fixing the operating member 70 to the curving portion 20, an arbitrary method can be selected according to the materials of the curving portion 20 and the operating member 70.

As recited herein throughout, the method of fixing can be effected by, for example, an adhesive made of a resin or a by heat fusion or the like. The instant disclosure and embodiments recited herein are not limited in the method of fixing or the substance/material used for fixing and those of skill in the art will select the appropriate fixing materials accordingly.

According to the number of insertion grooves 74 formed in the elongated member 10, two operating members 70 can be attached at positions offset from each other by 180° in the circumferential direction. As shown in FIG. 2A, the proximal part of each operating member 70 is connected to a rotating member 65 provided at the hand operating part 60. The rotating member 65 is pivotally supported by a rotating shaft (not shown) provided at the hand operating part 60. When the rotating member 65 is rotated in a direction of arrow A, the curving portion 20 curves into one direction (direction of the arrow A′ in FIG. 1). When the rotating member 65 is rotated in a direction of arrow B, the curving portion 20 curves into another direction (direction of the arrow B′ in FIG. 1).

As shown in FIGS. 2B and 3, in the elongated member 10, a covering member 75 that is so disposed as to cover the outer surface of the curving portion 20, the outer surface of the entry assisting portion 40, and the outer surface of the main body portion 50 can be provided. Covering member 75 can protect the respective parts of the elongated member 10. Furthermore, when causing various kinds of fluids to flow by using the elongated member 10, the occurrence of leakage of the fluid from the elongated member 10 can be favorably prevented. In addition, the efficiency of suction of a body fluid, secretion, and other biological fluids from the distal opening 21 of the curving portion 20 can also be enhanced. Moreover, it also becomes possible to prevent separation of the operating member 70 from the insertion groove 74 of the curving portion 20. The covering member 75 can be formed of, for example but not limited to, a hollow member in which a lumen extends along the axial direction.

The covering member 75 may be formed from materials such as but not limited to, polyolefin such as polyethylene (PE) and polypropylene (PP), polyester such as polyethylene terephthalate (PET), polyamide (PA), polyimide (PI), polyamide imide (PAI), silicone, polyurethane (PU), ethylene-vinyl acetate copolymer (EVA), polyvinyl chloride (PVC), fluorine-based resin such as polytetrafluoroethylene (PTFE), polyvinylidene fluoride (PVDF), and perfluoroalkoxy fluorine resin (PFA), and thermoplastic resin such as thermoplastic elastomer.

FIG. 6B is an enlarged diagram illustrating part of the groove 24 forming the curving mechanism. In groove 24, a predetermined gap g and a guide part 31 engaged with this gap g are formed. When the operating member 70 is operated, the guide part 31 moves in the gap g. This movement is transmitted along the longitudinal direction of the curving portion 20 and thereby the whole of the curving portion 20 curves. Providing a support point 32 that swingably supports the guide part 31 as shown in FIG. 6B makes it possible to favorably transmit the movement of the guide part 31 along the longitudinal direction.

As shown in FIG. 2B, entry assisting portion 40 is formed of a member in which the distal insertion hole 41 formed on the distal side, the proximal insertion hole 42 formed on the proximal side, and the lumen 43 communicating with the distal insertion hole 41 and the proximal insertion hole 42 are formed. The entry assisting portion 40 can be formed of, for example, a hollow member in which the lumen 43 extends along the axial direction.

As shown in FIG. 2B, the proximal part 22a of the curving portion 20 is inserted into the distal insertion hole 41 of the entry assisting portion 40. The curving portion 20 is press fitted into the distal insertion hole 41. For that reason, the curving portion 20 and the entry assisting 40 portion are fixed. The structure on the opposite end (proximal side) has the same configuration and relationship. The distal part 51a of the main body portion 50 is inserted into the proximal insertion hole 42 of the entry assisting portion 40. The main body portion 50 is so formed as to be fitted to the proximal insertion hole 42 along with the insertion. The main body portion 50 is fixed to the entry assisting portion 40 by the fitting. In this manner, the curving portion 20 and the main body portion 50 are mutually connected via the entry assisting portion 40. It is also possible to use an adhesive or the like in combination in order to enhance the fixing strength between the curving portion 20 and the entry assisting portion 40 and the fixing strength between the entry assisting portion 40 and the main body portion 50. In FIG. 2B, diagrammatic representation of the guide members 80 is omitted in order to clearly show the connection relationship among the curving portion 20, the entry assisting portion 40, and the main body portion 50.

As shown in FIG. 2B, a section of the lumen 43 of the entry assisting portion 40 along the axial direction is formed into a tapered shape whose diameter gradually decreases from the proximal side toward the distal side. The inner diameter of an end part 42a located on the side of the proximal insertion hole 42 in the lumen 43 of the entry assisting portion 40 is set substantially the same as the diameter of the lumen 53 of the main body portion 50. Furthermore, the inner diameter of an end part 41a located on the side of the distal insertion hole 41 in the lumen 43 of the entry assisting portion 40 is set substantially the same as the diameter of the lumen 23 of the curving portion 20. Therefore, flat joints having no step are formed between the proximal opening 22 of the curving portion 20 and the distal insertion hole 41 of the entry assisting portion 40, and between a distal opening 51 of the main body portion 50 and the proximal insertion hole 42 of the entry assisting portion 40.

Materials from which the entry assisting portion 40 may be formed may be the same material as that used to form the curving portion 20. However, as described herein below, the curving portion 20, the entry assisting portion 40, and the main body portion 50 can be so formed as to be different from each other in physical properties so that they may have desired and/or different functions. Therefore, the material for forming the entry assisting portion 40 can be selected according to the relationship with the characteristics of the materials used for the curving portion 20 and the main body portion 50.

As shown in FIGS. 2B and 3, the main body portion 50 can be formed of a hollow first object 55a and a hollow second object 55b that is so disposed as to cover the first object 55a. For the first object 55a, a tubular member formed of a metal coil can be used. For the second object 55b, a tubular member formed of a woven metal blade can be used. By using a coil as the first object 55a, the main body portion 50 is endowed with elasticity and sturdiness. By using a blade as the second object 55b, the main body portion 50 is endowed with rigidity and pushability. For example, it is also possible that the main body portion 50 is formed of a tubular member composed of a spiral tube formed by winding a strip plate into a spiral shape, a mesh net covering the spiral tube, and a resin outer coat covering the outer circumferential surface of the net. Furthermore, it is also possible to use the same material for the first object 55a and the second object 55b. Using the same material can enhance the bonding strength between both objects when both objects are bonded by using an adhesive or the like.

The material and configuration of the main body portion 50 are not particularly limited as long as it is endowed with pushability that allows transmission of a pushing force to the distal side. For example, it is also possible for the main body portion 50 is formed of a single-layer member without employing a multilayer structure. If the main body portion 50 is formed of a single-layer member, the following materials/substances can be used to form the single-layer member: polyvinyl chloride, polyethylene, polypropylene, cyclic polyolefin, polystyrene, poly(4-methylpentene-1), polycarbonate, acrylic resin, acrylonitrile-butadiene-styrene copolymer, polyester such as polyethylene terephthalate and polyethylene naphthalate, butadiene-styrene copolymer, polyamide (e.g. nylon 6, nylon 6•6, nylon 6•10, nylon 12), and various kinds rubber materials such as natural rubber, butyl rubber, isoprene rubber, butadiene rubber, styrene-butadiene rubber, and silicone rubber.

As shown in FIGS. 2A, 2B, and 3, in the main body portion 50, the distal part 51a at which the distal opening 51 is formed, a proximal part 52a at which a proximal opening 52 is formed, and the lumen 53 communicating with the distal opening 51 and the proximal opening 52 are formed.

As described above, the distal part 51a of the main body portion 50 is fitted to the proximal insertion hole 42 of the entry assisting portion 40. Furthermore, as shown in FIG. 2A, the proximal part 52a of the main body portion 50 is inserted into a distal opening 61 formed in the hand operating part 60 and is fitted along with this insertion. The main body portion 50 is fixed to the hand operating part 60 by this fitting.

As shown in FIG. 3, the guide members 80 extending from the distal side of the elongated member 10 to the proximal side can be disposed in contact with the inner surface of the main body portion 50. Disposing them in this manner can prevent narrowing of the usable space in the lumen 53 of the main body portion 50 due to the placing of the guide members 80. For example, it is also possible to form a predetermined groove extending along the axial direction in the inner surface of the main body portion 50 and dispose the guide member 80 in this groove.

As shown in FIG. 2A, the hand operating part 60 has the distal opening 61, into which the proximal end of the main body portion 50 is inserted, a space part 62 extending along the axial direction, support parts 63 that assist the operating members 70 in advancing and retreating, an insertion opening 64 formed at the proximal part, and the rotating member 65 used to carry out push/pull operation of the operating members 70.

The space part 62 of the hand operating part 60 is disposed coaxially with each of the lumen 23 of the curving portion 20, the lumen 43 of the entry assisting portion 40, and the lumen 53 of the main body portion 50. This space part 62 and the lumens 23, 43, and 53 of the respective portions form a working lumen for inserting a predetermined medical device, another medical instrument, or the like into the medical instrument 100. For example, when the procedure instrument 150 for medical use is delivered into the pulmonary airway P via the medical instrument 100 (see FIG. 9), the procedure instrument 150 is inserted into the inside of the hand operating part 60 via the insertion opening 64 formed in the hand operating part 60. The procedure instrument 150 is then inserted into the working lumen and its distal side is protruded from the distal opening 21 of the curving portion 20. The procedure instrument 150 can be thereby guided to a procedure-target site.

The rotating member 65 of the hand operating part 60 is rotatably attached in a state of being connected to the operating members 70. A concave-convex part on which a finger can be made to rest is formed on the surface of the rotating member 65. As shown in FIG. 1, for example, the curving operation of the curving portion 20 can be carried out by simple operation of rotating the rotating member 65 with use of a finger of one hand. The hand operating part 60 may be formed, for example, from a rigid plastic material or a metal material.

The dimensions, physical properties, and so forth of the respective parts of the elongated member will now be described.

Referring to FIG. 2A again, it is preferable that a length L1 of the curving portion 20 in the axial direction is at least 10 mm and at most 60 mm for example. Note that the length L1 of the curving portion 20 described here does not include the length of the part inserted into the distal insertion hole 41 of the entry assisting portion 40.

As shown in FIG. 7, when the elongated member 10 is moved toward the peripheral side, the elongated member 10 can be moved along the pulmonary airway P by moving the elongated member 10 in such a manner that it abuts against the inner wall surface of the pulmonary airway P. At the initial stage of the insertion, specifically for example while the elongated member 10 is being moved on the central side, the elongated member 10 can be moved smoothly by making the curving portion 20 of the elongated member 10 abut against (i.e., get caught on) the inner wall surface of the lobar bronchus P3 (around the second bifurcation) located on the central side. However, if the length L1 of the curving portion 20 is smaller than the diameter of the main bronchus P2 (around the first bifurcation), bending and inflection easily occur in the curving portion 20 in the midst of the movement of the curving portion 20 in the main bronchus P2 and therefore it is difficult for the curving portion 20 to reach the lobar bronchus P3. Therefore, the length L1 of the curving portion 20 is set to at least 10 mm, which is a general diameter of the main bronchus P2. To prevent the occurrence of the above-described problem, it is preferable that the length L1 of the curving portion 20 is at least 14 mm.

When pushing the elongated member 10 in order to further advance the curving portion 20 toward the peripheral side after the distal end of the curving portion 20 reaches the peripheral airway, the curving portion 20 can be moved toward the peripheral side by at most 60 mm due to the elongation of the peripheral airway. Therefore, by setting the upper limit of the length L1 of the curving portion 20 to approximately 60 mm, the curving portion 20 is prevented from being formed unnecessarily long with enabling of inclusion of a wider area of the peripheral area in procedure-target sites. The peripheral airway is elongated by at least 40 mm. Therefore, it is more preferable that the length L1 of the curving portion 20 is at most 40 mm in terms of preventing the curving portion 20 from being unnecessarily long.

Referring again to FIG. 2A, it is preferable that an outer diameter D1 of the curving portion 20 is at least 2.0 mm and at most 4.5 mm. As disclosed herein the outer diameter of the curving portion 20 means an outer diameter including the thickness of the covering member 75 if the covering member 75 is used, and also means an outer diameter including the thickness of a coating material to be described herein below, if the coating material is used.

The diameter of the respiratory bronchiole P7 in the pulmonary airway P is generally 0.5 to 1.0 mm (see FIG. 7). When the curving portion 20 is pushed into the respiratory bronchiole P7, the respiratory bronchiole P7 dilates to at most about 1.0 mm to 2.0 mm. Therefore, if the outer diameter D1 of the curving portion 20 is 2 mm, the curving portion 20 can be so moved as to abut against the inner wall surface of the respiratory bronchiole P7. Furthermore, referring to FIG. 2B, it is preferable that a diameter R1 of the lumen 23 of the curving portion 20 is at least 1 mm so that various kinds of medical devices and medical instruments can be inserted. For this reason, in consideration of the necessary minimum wall thickness of the curving portion 20, it is preferable that the outer diameter D1 of the curving portion 20 is at least 2 mm in terms of the relationship with the diameter R1 of the lumen 23.

The diameter of the peripheral airway of the pulmonary airway P is generally 2.0 mm. When the curving portion 20 is pushed into the peripheral airway, the peripheral airway dilates to at most about 4.5 mm. Therefore, if the outer diameter D1 of the curving portion 20 is 4.5 mm, the curving portion 20 can be moved so as to abut against the inner wall surface of the peripheral airway. For this reason, it is preferable that the upper limit of the outer diameter D1 of the curving portion 20 be set to 4.5 mm. Because the peripheral airway dilates to only about 3.5 mm in some cases, it is more preferable that the outer diameter D1 of the curving portion 20 is at most 3.5 mm in terms of the invasiveness.

Referring once again to FIG. 2A, it is preferable that a length L2 of the entry assisting portion 40 in the axial direction is at least 10 mm since the length L2 of the entry assisting portion 40 described here is the length from the distal end of a distal tapered part 45a to be described herein below to the proximal end of a proximal tapered part 45b to be described later (see FIG. 3).

When the curving portion 20 is moved in the peripheral airway or the respiratory bronchiole P7, a pushing force can be favorably transmitted to the curving portion 20 located on the distal side via the entry assisting portion 40 causing the entry assisting portion 40 to abut against the lobar bronchus P3 located on the central side (see FIG. 9) for example. However, if the length L2 of the entry assisting portion 40 is smaller than the diameter of the lobar bronchus P3, the entry assisting portion 40 is disposed sideways in the lobar bronchus P3. As a result, it becomes difficult to keep the abutting state of the entry assisting portion 40 against the lobar bronchus P3. For this reason, it is preferable that the length L2 of the entry assisting portion 40 be at least 10 mm, which is a general diameter of the lobar bronchus P3. To prevent the occurrence of the above-described problem more surely, it is more preferable that the length L2 of the entry assisting portion 40 be at least 15 mm.

As shown in FIG. 2A, it is preferable that a length L3 of the main body portion 50 along the axial direction is at least 230 mm and at most 800 mm. Note that the length of the main body portion 50 described here does not include the length of the part inserted into the proximal insertion hole 42 of the entry assisting portion 40 and the length of the part inserted into the space part 62 of the hand operating part 60.

Forming the main body portion 50 with the above-described length L3 enables the distal end of the curving portion 20 to surely reach a site on the peripheral side relative to the bronchus P4 when the elongated member 10 is introduced into the pulmonary airway P orally or nasally. Thus, it is possible to favorably perform various procedures in the peripheral airway including the bronchiole P5 and in the respiratory bronchiole P7.

In the elongated member 10 (as shown in FIG. 2A), a diameter D2 of the largest-outer-diameter part of the entry assisting portion 40 is set larger than the outer diameter D1 of the curving portion 20 and an outer diameter D3 of the main body portion 50. Therefore, as shown in FIG. 9, when the elongated member 10 is moved in the pulmonary airway, moving the curving portion 20 on the peripheral side can be carried out by retaining a state in which the entry assisting portion 40 abuts against the inner wall surface of the central side. Furthermore, when curving portion 20 on the peripheral side, the pushing force given from the proximal side is favorably transmitted to the side of the curving portion 20 via the entry assisting portion 40 and thus the pushability of the elongated member 10 is enhanced. The diameter D2 of the largest-outer-diameter part of the entry assisting portion 40 can be set to e.g. 3.5 mm to 5.5 mm and the outer diameter D3 of the main body portion 50 can be set to e.g. 3.0 mm to 5.0 mm.

Elongated member 10 is formed such that the rigidity of the entry assisting portion 40 is higher than that of the curving portion 20. This allows the pushing force given from the hand side to be efficiently transmitted to the side of the curving portion 20 via the entry assisting portion 40, which can further enhance the pushability of the elongated member 10. Furthermore, the elongated member 10 is formed so that the rigidity of the entry assisting portion 40 is higher than that of the main body portion 50. This can further enhance the operability of the elongated member 10 without impairing the pushability of the entry assisting portion 40.

Additionally, elongated member 10 is formed so that the elasticity of the entry assisting portion 40 is lower than that of the curving portion 20. This suppresses the elastic deformation of the entry assisting portion 40 and prevents impairing of the transmissibility of the pushing force via the entry assisting portion 40. Furthermore, the curving portion 20 is permitted to move on the peripheral side with elastic deformation, which enables the curving portion 20 to move more smoothly.

At the distal part of the entry assisting portion 40, the distal tapered part 45a, whose outer diameter gradually decreases toward the distal side, is formed. At the proximal part of the entry assisting portion 40, the proximal tapered part 45b, whose outer diameter gradually decreases toward the proximal side, is formed. By providing such tapered parts 45a and 45b, friction between the entry assisting portion 40 and the inner wall surface of the pulmonary airway P can be reduced and the pushability of the elongated member 10 can be enhanced. Furthermore, it is also possible to prevent the occurrence of damage or the like on the inner wall surface of the pulmonary airway P when pushing operation is carried out.

As shown in FIG. 2B, the elongated member 10 is so formed such that a diameter R3 of the lumen 53 of the main body portion 50 is larger than the diameter R1 of the lumen 23 of the curving portion 20. Therefore, when various kinds of procedure instruments 150 are introduced into the pulmonary airway P by using the elongated member 10, the procedure instruments 150 can be easily moved from the side of the main body portion 50 to the side of the curving portion 20. Additionally, when the elongated member 10 is applied to a suction device or a discharge device, the suction power can be enhanced or the discharge amount can be increased. The diameter R3 of the lumen 53 of the main body portion 50 can be set to e.g. 2.0 mm to 4.5 mm and the diameter R1 of the lumen 23 of the curving portion 20 can be set to e.g. 1.2 mm to 3.7 mm.

The surface of the curving portion 20 can be covered by a coating material having slipperiness. Enhancing the slipperiness of the curving portion 20 can further enhance the pushability of the elongated member 10. The coating material may, for example, be a material that forms a coating film on the surface of the curving portion 20 or a material formed as a film material covering the surface of the curving portion 20. In the case of providing the covering member 75 on the curving portion 20, a configuration in which the coating material is applied on the covering member 75 can be employed. The coating material provided on covering member 75 may be a hydrophilic material or a hydrophobic material.

Examples of the hydrophilic material include cellulose-based polymer substance, polyethylene oxide-based polymer substance, maleic anhydride-based polymer substance (e.g. maleic anhydride copolymer such as methyl vinyl ether-maleic anhydride copolymer), acrylamide-based polymer substance (e.g. polyacrylamide and block copolymer of poly(glycidyl methacrylate)-dimethylacrylamide (PGMA-DMAA)), water-soluble nylon, polyvinyl alcohol, and polyvinylpyrrolidone.

In many cases, such a hydrophilic material exerts lubricity through moistening (water absorption) and reduces the frictional resistance (sliding resistance) against the inner wall of a wet biological organ. This enhances the slidablity of the elongated member 10 and makes the operability more excellent.

Examples of the hydrophobic material include polyamide, polyimide, polyurethane, polystyrene, silicone resin, fluorine-based resin (PTFE, ETFE, etc.), and composite materials of them. When such a hydrophobic material is used, the same effects as those by the above-described hydrophilic material can be exerted.

It is also possible to employ a configuration in which the inner surface of the lumen 23 of the curving portion 20 is covered by the coating material. This configuration makes it possible to smoothly perform the insertion of the procedure instrument 150 into the curving portion 20 or of the withdrawal of it from the curving portion 20.

The elongated member 10 is so formed that the torque transmissibility of the main body portion 50 is higher than that of the curving portion 20. By employing this configuration, when rotational operation or twist operation is carried out in a state in which the curving portion 20 abuts against the inner wall surface of the peripheral side of the pulmonary airway P, the action of the curving portion 20 following this operation can be suppressed. This can favorably prevent the occurrence of damage or the like on the pulmonary airway P. Further, because the torque transmissibility of the main body portion 50 is retained, the main body portion 50 can be moved with rotational operation or twist operation and thus the lowering of the operability of the elongated member 10 can be suppressed.

An example of a method of using the elongated member 10 according to the present embodiment will now be described.

Referring to FIG. 7, a part on the distal side of the medical instrument 100, specifically the distal end of the curving portion 20 of the elongated member 10, is inserted into the pulmonary airway P from the central side of the pulmonary airway P. The insertion can be carried out orally or nasally for example.

After the distal end of the curving portion 20 reaches the first bifurcation, the curving portion 20 is curved as shown in FIG. 7 to orient the distal end of the curving portion 20 toward a desired direction. The work of moving the elongated member 10 can be performed while the course of the elongated member 10 is appropriately decided by using an imaging unit such as an endoscope. In this manner, an image whose field of view includes the distal side of the elongated member 10 can be acquired by protruding the endoscope from the distal opening 21 of the curving portion 20 through the working lumen formed inside the medical instrument 100. The elongated member 10 can be moved along the desired course by repeating the protrusion of the endoscope from the distal opening 21 of the curving portion 20, check of the status of the distal side of the elongated member 10, and the forward movement of the elongated member 10.

With reference to FIG. 8, elongated member 10 is further pushed ahead toward the peripheral side of the pulmonary airway P. At this time, as a result of the contact of the main body portion 50 and the curving portion 20 of the elongated member 10 with the inner wall surface of the pulmonary airway P in the respective areas of the pulmonary airway P, the elongated member 10 can be so moved as to follow the pulmonary airway P.

Referring to FIG. 9, the elongated member 10 is pushed ahead until the curving portion 20 of the elongated member 10 reaches the respiratory bronchiole P7 located on the peripheral side. When the elongated member 10 reaches the respiratory bronchiole P7, the curving portion 20 abuts against the inner wall surface of the respiratory bronchiole P7. Thus, it becomes possible to move the curving portion 20 along the inner wall surface of the respiratory bronchiole P7. While the curving portion 20 is being moved in the respiratory bronchiole P7, the entry assisting portion 40 of the elongated member 10 is kept abutting against the inner wall surface of the lobar bronchus P3 for example. This allows the pushing force to be favorably transmitted to the side of the curving portion 20 via the entry assisting portion 40, enabling the curving portion 20 to move more smoothly.

After the distal end of the curving portion 20 reaches a desired position in the pulmonary airway P, a procedure instrument 150 is introduced via the elongated member 10. By using the procedure instrument 150, various kinds of procedures can be performed from a position close to a procedure-target site existing on the peripheral side. The procedure instrument 150 may include, for example, any of the following instruments: biopsy device, ultrasonic diagnosis device, microcatheter, ablation device, cryocatheter, radio-frequency ablation catheter, microwave ablation catheter, and PDT probe. When an endoscope is used in combination, the work of interchanging the endoscope with the procedure instrument 150 is performed appropriately.

As described above, inside the elongated member 10, a step is not formed at the connection part as the joint across which the curving portion 20 and the main body portion 50 are connected. Therefore, the procedure instrument 150 can be moved smoothly in the working lumen. Furthermore, there is no fear of, for example, the procedure instrument 150 breaking by getting caught on a step. When the elongated member 10 is applied to a medical device for suction of a body fluid, mucus, etc., or discharge of a medication, the fluid can be made to flow smoothly since no step is formed inside the elongated member 10.

After a predetermined procedure is performed, the procedure instrument 150 is withdrawn from the medical instrument 100. When withdrawing the procedure instrument 150, the procedure instrument 150 does not get caught on a step inside the elongated member 10 and thus can be smoothly moved.

After the procedure instrument 150 is withdrawn from the living body, the work of withdrawing the medical instrument 100 from the living body is performed to end the manipulation. The manipulation may be continued by carrying out operation of moving the elongated member 10 again toward another procedure-target site through repetition of the same process.

As above, according to the present embodiment, the elongated member 10 can be formed by connecting the curving portion 20 and the main body portion 50 that are different from each other in the lumen diameter, outer diameter, and physical properties. Therefore, by selecting members suitable for the dimensions and physical properties of the respective parts of the elongated member 10, the following elongated member 10 for medical use suitable to be used in the pulmonary airway P can be provided. Specifically, the distal side is given a configuration that has a small diameter and is excellent in flexibility and the proximal side is endowed with pushability. Moreover, the lumen diameter on the proximal side is set large. Furthermore, in the entry assisting portion 40 used to connect the curving portion 20 and the main body portion 50, the lumen 43 is formed, whose diameter continuously changes from the proximal side toward the distal side according to the difference between the diameter R1 of the lumen 23 of the curving portion 20 and the diameter R3 of the lumen 53 of the main body portion 50. Therefore, the curving portion 20 and the main body portion 50 can be connected without forming a step at the connection part as the joint of the curving portion 20 and the main body portion 50. This can provide the elongated member 10 for medical use with which the smooth movement of various kinds of procedure instruments 150, fluids, and so forth in the working lumen is realized.

Furthermore, the elongated member 10 is so configured that the curving portion 20 and the main body portion 50 can be connected by simply inserting the proximal part 22a of the curving portion 20 into the distal insertion hole 41 of the entry assisting portion 40 and inserting the distal part 51a of the main body portion 50 into the proximal insertion hole 42 of the entry assisting portion 40. This facilitates manufacturing and reduces the manufacturing costs.

Moreover, the tapered parts 45a and 45b are formed at the distal part and the proximal part, respectively, of the entry assisting portion 40. Therefore, when the elongated member 10 is moved in the pulmonary airway P, friction between the entry assisting portion 40 and the inner wall surface of the pulmonary airway P can be reduced and the pushability of the elongated member 10 can be enhanced. It is additionally possible to prevent the occurrence of damage on the inner wall surface of the pulmonary airway P when the elongated member 10 is pushed.

Furthermore, the outer surfaces of the respective parts of the elongated member 10 are covered by the covering member 75. Therefore, the respective parts of the elongated member 10 can be protected and breakage in the elongated member 10 can be prevented. In addition, when various kinds of fluids flow through the elongated member 10, the occurrence of the leakage of the fluid from the elongated member 10 is favorably prevented.

Accordingly it is possible to provide the connecting member 40 that is used to assemble the elongated member 10, including the curving portion 20 and the main body portion 50 as its constituent elements, and to allow the respective members 20 and 50 to be connected without forming a step on the inner surface of the connection part across which the respective members 20 and 50 are connected.

### MODIFICATION EXAMPLE

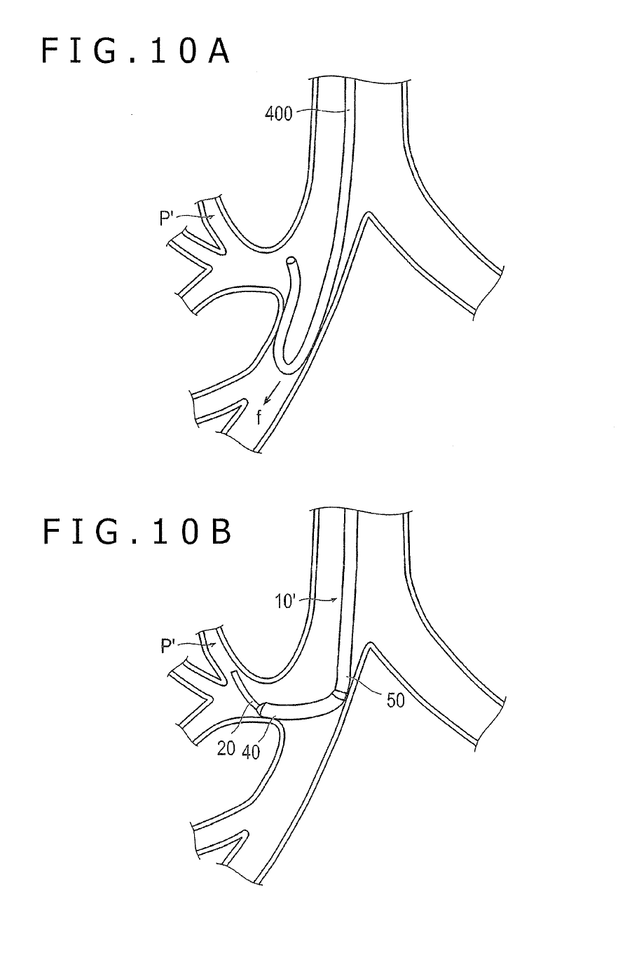

In FIG. 10A, when a medical device 400 such as a related-art elongated member for medical use or a thin bronchoscope, is inserted into a superior lobar bronchus P′ in a procedure using the medical device 400, the medical device 400 needs to be made to bend (meander) to a large extent. In addition, the direction in which the force is applied (indicated by an arrow f in the diagram) is opposite to the traveling direction of the medical device 400. Therefore, the advancing force is not transmitted to the distal end of the medical device 400 and in some cases the medical device 400 does not move as planned. In particular, when the distal end of the medical device 400 is inserted into a right superior lobar bronchus and is moved ahead from segmental bronchi (third bifurcation to fourth bifurcation) to sub-segmental bronchi (fourth bifurcation to fifth bifurcation), the inflection of the longitudinal part of the medical device 400 occurs in an intermediate bronchial trunk, so that the medical device 400 becomes a J-shape as shown in the diagram and in such case drops down with this shape. In a bronchoscopic image, when the distal end enters the right superior lobar bronchus and is pushed ahead directly, the entrance of the superior lobar bronchus suddenly becomes remote and the right principal bronchus comes into view.

In an elongated member 10′ according to a modification example shown in FIG. 10B, the length of the entry assisting portion 40 is set longer than the diameter of the intermediate bronchial trunk. Furthermore, the elasticity of the entry assisting portion 40 is set comparatively low. Instead of setting the elasticity low, the rigidity and outer diameter may be set comparatively large for example. According to this elongated member 10′, when a procedure with use of this elongated member 10′ is performed, the entry assisting portion 40 can be made to get caught on the inner wall surface of the intermediate bronchial trunk and thus the occurrence of the dropping of the elongated member 10′ in the intermediate bronchial trunk can be favorably prevented. Therefore, an elongated member 10′ that is superior in operability can be provided.

Although the elongated member according to the present disclosure is described above through the embodiment and the modification example, the present disclosure is not limited only to the above-described respective embodiments and can be appropriately modified based on the description of the scope of claims.

For example, the configuration of the curving portion 20 provided in the elongated member 10 is not limited to one in which a curving action is made based on the curving mechanism formed with grooves and rather may be one formed by juxtaposing tubular members such as joint rings in the longitudinal direction. The configuration of the hand operating part 60 can be changed as long as it can curve the curving portion 20. It is also possible to use the elongated member 10 alone as a medical instrument without providing the hand operating part 60. The procedure instruments 150 for medical use and the contents of procedures that are exemplified are one example and it is possible to perform various kinds of procedures with use of a procedure instrument other than those exemplified herein.

For example, it is also possible that the distal part of the elongated member 10 is not formed as a configuration that can curve but formed of a predetermined bendable tube or the like endowed with flexibility.

The elongated member is not limited to a configuration in which two members are connected and it is also possible to form it by connecting three or more members for example. In such a configuration, by using the connecting member, a step can be prevented from being formed at the parts as the joints among the respective connected members.

For example, at the distal side of the elongated member 10, a balloon can be disposed that is publicly known in the medical field and is so formed as to be capable of inflation and deflation through injection and discharge of a fluid. By inflating the balloon in administration of a medication or the like via the elongated member 10, the administration work can be performed in a state in which the position of the elongated member 10 is fixed and settled. Thus, the medication administration can be efficiently performed. Furthermore, it becomes possible to occlude the peripheral side of the pulmonary airway P by the balloon and administer a medication or the like to the distal side relative to the occluded part. Therefore, local administration of the medication is also enabled.

When using the balloon, the balloon may be so disposed as to cover the covering member 75 or may be disposed directly on the outer surface of the elongated member 10. The balloon is connected to a fluid introduction lumen in such a manner that a fluid can be introduced to the balloon from the hand side, and the balloon is inflated by introduction of a fluid from this fluid introduction lumen. The fluid introduction lumen may be disposed outside the elongated member 10 or may be disposed inside it. The balloon is formed of a material that expands and contracts, such as silicone. As a result, the balloon is brought into close contact with a biological lumen when being inflated and operates in a state in which the elongated member 10 is fixed at a predetermined position. However, the balloon is not limited in this manner and a material that does not expand and contract, such as nylon or polyethylene, may be disposed in a folded state. If the balloon is formed of such a material, dilation of a narrowed part generated in a bronchus is also enabled.

It should be understood by those skilled in the art that various modifications, combinations, sub-combinations and alterations may occur depending on design requirements and other factors insofar as they are within the scope of the appended claims or the equivalents thereof.

While illustrative and presently preferred embodiments of the present invention have been described in detail herein, it is to be understood that the inventive concepts may be otherwise variously embodied and employed without departing from the spirit and scope of the invention. The appended claims are intended to be construed to include such variations and equivalents.

What is claimed is:

1. An elongated member for medical use for use in performing a procedure in a pulmonary airway, the elongated member comprising:
a first member disposed on a distal side of said elongated member;
a second member in which a second lumen is formed;
a connecting member having a distal insertion hole into which a proximal part of the first member is insertable, a proximal insertion hole into which a distal part of the second member is insertable, and a third lumen that communicates with the distal insertion hole and the proximal insertion hole.

2. The elongated member according to claim 1, wherein the first member has a first lumen extending along an axial direction in the first member.

3. The elongated member according to claim 1, wherein the second lumen extends along the axial direction and has a larger inner diameter than the first lumen.

4. The elongated member according to claim 3, wherein the second member has a larger outer diameter than the first member and being disposed on a proximal side of the first member.

5. The elongated member according to claim 1, wherein the connecting member connects the first member and the second member.

6. The elongated member according to claim 1, wherein the third lumen has a diameter that gradually decreases from the proximal side toward the distal side from the diameter of the second lumen to the diameter of the first lumen.

7. An elongated member for medical use for use in performing a procedure in a pulmonary airway, the elongated member comprising:
a first member disposed on a distal side, a first lumen extending along an axial direction formed in the first member;
a second member in which a second lumen is formed, the second lumen extends along the axial direction and has a larger inner diameter than the first lumen is formed, the second member having a larger outer diameter than the first member and being disposed on a proximal side of the first member; and
a connecting member having a distal insertion hole into which a proximal part of the first member is insertable, a proximal insertion hole into which a distal part of the second member is insertable, and a third lumen that communicates with the distal insertion hole and the proximal insertion hole, a diameter of the third lumen gradually decreasing from the proximal side toward the distal side from the diameter of the second lumen to the diameter of the first lumen, the connecting member connecting the first member and the second member.

8. The elongated member according to claim 7, wherein
the proximal part of the first member is formed so as to be capable of being connected to the distal insertion hole of the connecting member through fitting, and
the distal part of the second member is formed so as to be capable of being connected to the proximal insertion hole of the connecting member through fitting.

9. The elongated member according to claim 8, wherein
a distal tapered part whose outer diameter gradually decreases toward the distal side is formed at a distal part of the connecting member, and
a proximal tapered part whose outer diameter gradually decreases toward the proximal side is formed at a proximal part of the connecting member.

10. The elongated member according to claim 7, further comprising
a covering member that is disposed so as to cover an outer surface of the first member, an outer surface of the connecting member, and an outer surface of the second member.

11. A connecting member to assemble an elongated member for medical use that is used to perform a procedure in a pulmonary airway by connecting a proximal side of a first member in which a first lumen extending along an axial direction is formed to a second member in which a second lumen that extends along the axial direction and has a larger inner diameter than the first lumen is formed, the second member having a larger outer diameter than the first member, the connecting member comprising:
a distal insertion hole into which a proximal part of the first member is insertable;
a proximal insertion hole into which a distal part of the second member is insertable; and
a third lumen that communicates with the distal insertion hole and the proximal insertion hole, an inner diameter of the third lumen gradually decreasing from the proximal side toward a distal side from the inner diameter of the second lumen to the inner diameter of the first lumen.
